(19) United States
(12) Patent Application Publication
(10) Pub. No.: US 2021/0401619 A1
(43) Pub. Date: Dec. 30, 2021

(54) INTRAOCULAR IRRIGATION FLUID TEMPERATURE MODULATOR SYSTEM AND METHOD OF USING THE SAME

(71) Applicant: OPTICGON LLC, Flushing, NY (US)

(72) Inventor: IAN H. CHAN, Flushing, NY (US)

(21) Appl. No.: 17/363,533

(22) Filed: Jun. 30, 2021

Related U.S. Application Data
(60) Provisional application No. 63/046,025, filed on Jun. 30, 2020.

(51) Int. Cl. A61F 9/007; A61F 7/08; A61F 7/00; A61M 3/02

(52) U.S. Cl. CPC A61F 9/007; A61F 7/08; A61F 7/0085; A61M 3/02; A61M 2210/0612; A61F 2007/0094; A61F 2007/083; A61F 2007/0095; A61F 2007/0077; A61F 2007/0054; A61M 2205/3368

(57) ABSTRACT

The present invention is directed to the field of ophthalmological surgery and pertains to a system to modulate the intraocular irrigation solution for the anterior and/or posterior chamber of the eye to body temperature during an ophthalmological surgery.

### RELATED PATENT APPLICATIONS

This application claims benefit to U.S. Provisional Application No. 63/046,025, filed Jun. 30, 2020, and is incorporated herein by reference in its entirety.

### FIELD

This disclosure relates to the field of ophthalmological surgery systems. In particular, this disclosure relates to intraocular irrigation fluid temperature modulator systems, and methods of their use in delivering ocular fluids to the anterior and/or posterior chambers of the eye of a patient during ophthalmological procedures.

### BACKGROUND

The following includes information that may be useful in understanding the present invention. It is not an admission that any of the information, publications or documents specifically or implicitly referenced herein is prior art, or essential, to the presently described or claimed inventions. All publications and patents mentioned herein are hereby incorporated by reference in their entirety.

During ophthalmological surgeries, irrigation fluid having about the same isotonicity as natural eye fluid is often presented to the eye of a subject so as to maintain fluid pressure during the course of the surgery. Common irrigation fluid used in intraocular surgery includes variations of balance salt solution and sometimes with supplementary glutathione and sugar, such as the Alcon BSS or BSS Plus solution. The irrigation fluid simulates the ionic concentration, pH and osmolarity of the “normal” extracellular environment. The irrigation fluid bottle is usually stored in room temperature at about 20-22° C. During surgery with active infusion, due to fluid flow and turbulence, the irrigation fluid temperature will fall below room temperature to roughly 10-15° C. This fluid is directly in contact with ocular vascular tissues such as the retina, choroid or iris, thus cooling the ocular tissue and associated blood vessels and circulation to below room temperature. These factors lead to variable clinical outcomes for individuals having ophthalmological surgeries.

In light of the above, there remains a need for accurately controlling ophthalmological irrigation fluid temperature without affecting the delivery pressure.

### SUMMARY

The inventions described and claimed herein have many attributes and aspects including, but not limited to, those set forth or described or referenced in this Brief Summary. It is not intended to be all-inclusive and the inventions described and claimed herein are not limited to or by the features or embodiments identified in this Brief Summary, which is included for purposes of illustration only and not restriction.

This disclosure provides for intraocular irrigation fluid temperature modulator systems for delivering ocular fluids to the eye of a patient during ophthalmological procedures. In one aspect, the intraocular irrigation fluid temperature modulator system comprises one or a plurality of heating elements configured to be in thermal contact with a bottle. In some aspects, the heating element is a heating tape. In some aspects, the bottle comprises glass, metal, or a combination thereof. The bottle is non-compressible, in that the material will not bend or deform during an ophthalmological procedure. In some aspects, the bottle comprises an enclosed glass cylinder with one or a plurality of openings. In some aspects, the bottle comprises an enclosed glass cylinder with one or a plurality of openings wherein the glass cylinder is surrounded by metal. In some aspects, the bottle comprises an ocular irrigation fluid and a gas. In some aspects, the bottle comprises one or a plurality of one-way pressure valves, which are optionally independently connected to a gas. In some aspects, the gas is selected from air, sulfur hexafluoride, nitrous oxide, oxygen, nitrogen, or combinations thereof. In some aspects, the heating element comprises embedded heating coils. In some aspects, the system is configured to be portable. In some aspects, the system is configured to be wireless. In some aspects, this disclosure provides for a system for modulating the temperature of an intraocular irrigation fluid comprising: (a) a heating element, (b) a bottle, wherein the heating element closely conforms to the exterior contour of the irrigation fluid bottle. In some aspects, the system for modulating the temperature of an intraocular irrigation fluid further comprises a means to conform the heating element to the exterior contour of the irrigation fluid bottle. In some aspects, the means to conform the heating element to the exterior contour of the irrigation fluid bottle is selected from a reversible glue, a buckle, or a Velcro wrap.

In some aspects, the intraocular irrigation fluid temperature modulator system comprises an intraocular irrigation fluid temperature modulator system described herein and control electronics. In some aspects, the control electronics further comprise a thermocouple positioned to measure the temperature of the external surface of the bottle. The control electronics modulate the voltage and/or current applied to the heating element so as to modulate the measured temperature within a selected range. In some aspects, the control electronics further comprises a receiving device which receives a wireless signal from a control emitter device which identifies the selected temperature range to which the heating element is to modulate to. In some aspects, the control electronics comprises a thermometer or temperature display to indicate about the temperature measured by the thermocouple. In some aspects, the control electronics further comprises a means to electronically disconnect and optionally reconnect, the applied voltage and/or current to the heating element. In some aspects, the means to electronically disconnect and optionally reconnect, the applied voltage and/or current to the heating element comprises a manual trigger button. In some aspects, the control electronics further comprises an audio alarm which activates when the measured temperature is outside of the selected temperature range. In some aspects, the control electronics further comprises an electronic circuit which measures the temperature and selects to apply voltage and/or current to the heating element when the measured temperature is less than a selected temperature range, or applies no voltage and/or current to the heating element when the measured temperature is higher than a selected temperature range. In some aspects, the control electronics further comprise a rechargeable battery pack. The rechargeable battery pack can provide current and/or voltage to the one or plurality of heating elements so as to modulate the temperature of the bottle. In some aspects, the battery pack is rechargeable. In some aspects, the rechargeable battery pack comprises a lithium ion battery. In some aspects, the rechargeable battery pack comprises a port for external power. In some aspects, the port for external power is a USB (Universal Serial Bus) connection. In some aspects, the electronics control system is configured to receive input from a plurality of temperature sensors. The electronics control system can electronically disconnect the power supply source from the heating element when the plurality of temperature sensors indicates a difference in temperature when the difference between any two temperature sensors is greater than a selected temperature difference.

In some aspects, the system for modulating the temperature of an intraocular irrigation fluid further comprises an insulator which is configured around a portion or all of the bottle.

In some embodiments, the heating element comprises a water-resistant material such as polymer, fabric, polyester, or silicone. In some aspects, the heating element is one or a plurality of heating coils. In some aspects, the heating coils are configured to be in a “W” pattern periodically about the circumference of the bottle.

In some aspects, the intraocular irrigation fluid temperature modulator system further comprises a transparent window to allow direct visualization of the contents of the bottle. In some aspects, the transparent window is configured to enable visualization of the content fill volume in the bottle.

In another aspect, this disclosure provides for a method of using the intraocular irrigation fluid temperature modulator system described herein to present intraocular irrigation fluid at a selected temperature range to the eye of a subject during an intraocular surgery. In some aspects, the intraocular surgery can include or exclude: cataract surgery, vitrectomy surgery, glaucoma surgery, corneal surgery and other intraocular surgical procedures in the presence of intraoperative hemorrhage or risk of intraoperative hemorrhage.

### BRIEF DESCRIPTION OF THE DRAWINGS

The drawings form part of the present specification and are included to further demonstrate certain aspects of the embodiments described herein. These embodiments may be better understood by reference to one or more of the following drawings in combination with the detailed description.

FIG. 1 shows the effects of temperature on bleeding times, as measured for the coagulation cascade. FIG. 1A shows the prothrombin time as a function of temperature. FIG. 1B shows the activated partial thromboplastin time as a function of temperature.

FIG. 2 shows a conventional vitrectomy machine setup with the Alcon Constellation and a reference infusion bottle. The infusion bottle is hanging to the left side of the machine and is exposed to room temperature during surgery. There is no bottle temperature control in this reference device.

FIG. 3 depicts a representative embodiment of the intraocular irrigation fluid temperature modulator system described herein. A band 10 wraps around the clear infusion bottle 20. The battery pack 30 is affixed to the band, and optionally is configured to be within a pocket in the band. The electronic control unit with temperature sensor and display 40 affixed to the exterior surface of the band 50. The interior surface of the band (not shown) is in thermal contact with the infusion bottle 20. A securing device (e.g., a Velcro strip) 60 connects the band 10 around the infusion bottle 20. An optional transparent window 70 within the band allows for the visual assessment of the fill volume of the infusion bottle 20. The top surface of the infusion bottle comprises a central surface 80a and a posterior surface 80b, each of which independently can be covered with a warming pad, thermal insulating pad, or null, so as to enhance the efficacy of thermal control of the infusion bottle 20 by the warming band 10. In some embodiments, the bottle further comprises one or a plurality of temperature sensors 90 (depicted as small squares) configured to be in thermal contact with the infusion bottle 20. The bottle optionally comprises a portion of the exterior surface which is not covered by the band 100. Cross-surface 110 (shown as a grid) depicts the x-y axes only and is not part of the embodiment.

FIG. 4 depicts a flattened perspective of one representative embodiment of the heating element of the intraocular irrigation fluid temperature modulator system described herein. A band 10 is layed out flat in the absence of an irrigation bottle, with the grid indicating the x-y axes only and is not part of the embodiment. A heating coil 120 is optionally configured to be dispersed about uniformly across the band 10.

FIG. 5 is a picture of a representative embodiment of the intraocular irrigation fluid temperature modulator system described herein, where the intraocular irrigation fluid temperature modulator system is wrapped about an ocular fluids irrigation bottle.

FIG. 6 is a picture of a representative embodiment of the intraocular irrigation fluid temperature modulator system described herein, where the intraocular irrigation fluid temperature modulator system is opened, showing the interior surface. An interface to a power supply can also be seen.

FIG. 7 is a picture of a representative embodiment of the intraocular irrigation fluid temperature modulator system described herein, where the intraocular irrigation fluid temperature modulator system is opened, showing the exterior surface. The digital temperature display can be seen. An interface to a power supply can also be seen.

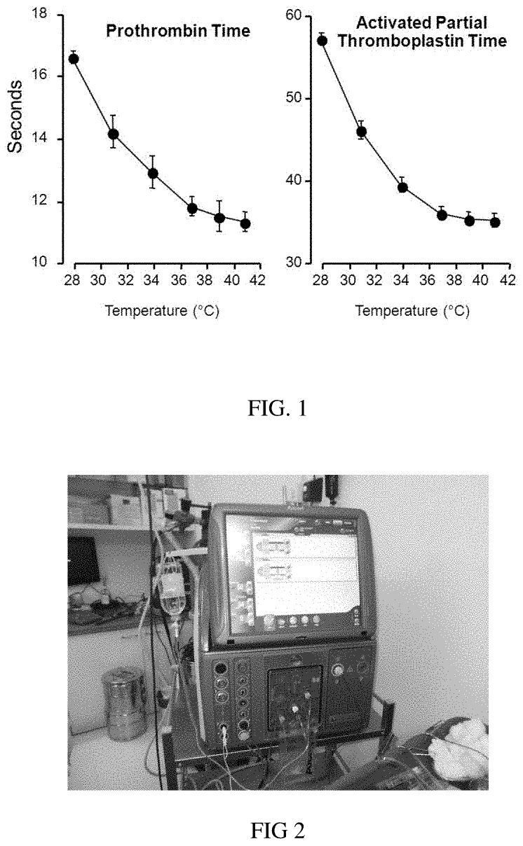

### DETAILED DESCRIPTION

This description of the exemplary embodiments is intended to be read in connection with the accompanying drawings, which are to be considered part of the entire written description. The drawing figures are not necessarily to scale and certain features may be shown exaggerated in scale or in somewhat schematic form in the interest of clarity and conciseness. In the description, relative terms such as “horizontal,” “vertical,” “up,” “down,” “top” and “bottom” as well as derivatives thereof (e.g., “horizontally,” “downwardly,” “upwardly,” etc.) should be construed to refer to the orientation as then described or as shown in the drawing figure under discussion. These relative terms are for convenience of description and normally are not intended to require a particular orientation. Terms including “inwardly” versus “outwardly,” “longitudinal” versus “lateral” and the like are to be interpreted relative to one another or relative to an axis of elongation, or an axis or center of rotation, as appropriate. Terms concerning attachments, coupling and the like, such as “connected” and “interconnected,” refer to a relationship wherein structures are secured or attached to one another either directly or indirectly through intervening structures, as well as both movable or rigid attachments or relationships, unless expressly described otherwise. The term “operatively connected” is such an attachment, coupling or connection that allows the pertinent structures to operate as intended by virtue of that relationship.

Disclosed are components that can be used to perform the disclosed methods and systems. The combinations, subsets, interactions, groups, etc. of these and all other components, along with their collective combinations and permutation may not be explicitly disclosed for all systems and associated methods. This applies to all aspects of this application including, but not limited to, steps in disclosed methods. Thus, if there are a variety of additional steps that can be performed it is understood that each of these additional steps can be performed with and specific embodiment or combination of embodiments of the disclosed methods and systems.

Conventional methods of attempting to modulate the temperature of intraocular irrigation fluids fails to adequately prevent bleeding in the eye of a subject because of engineering faults. A pre-warmed irrigation bottle immediately used in an ophthalmological surgery will result in a fast temperature equilibrium with room temperature soon after surgery begins, as some of the complex ophthalmological surgeries may take over 2 hours to complete.

### Definitions

As used herein, the recitation of a numerical range for a variable is intended to convey that the invention may be practiced with the variable equal to any of the values within that range. Thus, for a variable that is inherently discrete, the variable can be equal to any integer value of the numerical range, including the end-points of the range. Similarly, for a variable, which is inherently continuous, the variable can be equal to any real value of the numerical range, including the end-points of the range. As an example, a variable which is described as having values between 0 and 2, can be 0, 1 or 2 for variables which are inherently discrete, and can be 0.0, 0.1, 0.01, 0.001, or any other real value, for variables which are inherently continuous.

As used herein, the term “subject” or “subject in need thereof” refers to humans as well as non-human animals, such as domesticated mammals including, without limitation, pigs, cats, dogs, and horses. The subject is typically a mammal, more typically a human. However, the invention is not limited to the angiography imaging of humans and is applicable to veterinary uses.

As used herein, the term “irrigation fluid” refers to an aqueous solution presented to one or a plurality of chambers of the eye of a subject during an ophthalmological procedure. In one embodiment, the irrigation fluid is Alcon BSS (balanced salt solution) Irrigation Solution (Alcon (Fort Worth, Tex.), Akorn (Lake Forest, Ill.), B. Braun or Baxter Healthcare. In some embodiments, Alcon BSS Sterile Irrigating Solution is a sterile balanced salt solution, each mL containing sodium chloride (NaCl) 0.64%, potassium chloride (KCl) 0.075%, calcium chloride dihydrate (CaCl2.2H2O) 0.048%, magnesium chloride hexahydrate (MgCl2.6H2O) 0.03%, sodium acetate trihydrate (C2H5NaO2.3H2O) 0.39%, sodium citrate dihydrate (Na3C6H5O7.2H2O) 0.17%, sodium hydroxide and/or hydrochloric acid (to adjust pH), and water for injection. The pH is approximately 7.5. The osmolality is approximately 300 mOsm/Kg. In some embodiments, the irrigation fluid further comprises an additive selected from glutathione, glucose, bicarbonate, and combinations thereof.

Methods

There are three main temperatures during cataract surgery: the refrigerator where ophthalmic solutions are commonly stored (38° F., 3° C.), the operating room ambient temperature (68° F., 20° C.), and the physiologic body temperature (98° F., 37° C.). The optimal temperature for blood clot formation is around body temperature of 37° C. (Maxwell S. et al. Optimum Temperature of Formation of a Blood Clot. Nature v141 n3563:287-288; Olbrich S E et al. Effect of Temperature and Ration on Blood Clotting Time of Heat Tolerat and Cold Tolerant Cattle; Lawrence M J et al. The effects of Temperature on Clot Microstructure and Strength in Healthy Volunteers. Anesthesia and analgesia 2016 January; 122(1): 21-6). Hypothermia is a risk factor for decreased blood clot formation and increased bleeding risk during surgery. The quality of the clot as well as the clotting time is negatively affected by temperature (Valeria C R et al Nonsurgical bleeding diathesis in anemia thrombocytopenic patients: role of temperature, red blood cells, platelets and plasma-clotting proteins. Transfusion. 2007 October; 47(4) suppl: 206S-248S; Reynolds L et al. Perioperative complications of hypothermia. Best Pract Res Clin Anaesthesiol 2008; 22: 645-657; Buggy D J et al. Thermoregulation, mild perioperative hypothermia and postanaesthetic shivering. Br Anaesth 2000; 84:615-628). Temperature not only affects platelet function, but bleeding times is 4 times longer in room temperature of 22° C. versus 37° C. by altering the functioning of the factors in the coagulation cascade. Hypothermia poses significant risk of bleeding due to prolonged clotting time (Valeria et al. Effects of temperature on bleeding and clotting time. Crit Care Med 1995 Apr. 23(4):698-704; Simpson S et al. The effect of temperature on blood coagulation time. Quarterly Journal of Experimental Physiology 1916 Vol 10 Issue 2). Even mild hypothermia can lead to increased risk of intraoperative bleeding and it is very important to avoid hypothermia to minimize intraoperative blood loss (Rajagopalan S, et al. The effects of mild perioperative hypothermia on blood loss and transfusion requirement. Anesthesiology 2008; 108:71-77; Schmied H et al. Mild hypothermia increases blood loss and transfusion requirements during total hip arthroplasty. Lancet 1996; 347: 289-292; Shah A et al. Strategies to minimize intraoperative blood loss during major surgery. British Journal of Surgery Vol 107, Issue 2).

The inventors have surprisingly discovered that, in contrast to conventional intraocular surgical methods, cooled intraocular irrigation solutions are not presented to the eye of a subject at an ideal temperature. The inventors have discovered systems and methods for presenting intraocular irrigation fluid used during ophthalmological surgeries (which can include or exclude vitrectomy or phacoemulsification) to warm the intraocular irrigation fluid to a target temperature range near physiological body temperature wherein the body's clotting factors will function at an optimal condition. This will decrease the risk of bleeding so as to allow for better control of intraoperative bleeding. This is especially critical for intraocular surgeries of patients with a high risk of vitreous or choroidal hemorrhage (which can include or exclude patients with diabetic retinopathy, hypertensive retinopathy, proliferative vitreoretinopathy, hemophilia, a history of multiple ocular surgeries, retinal vein or arterial occlusions, neovascular glaucoma and complex or tractional retinal detachment or presence of retinal or choroidal neovascularization, hypertension, anemia, lymphoma, cancer, ocular ischemic syndrome, respiratory disease, and neovascular glaucoma).

During certain surgical scenarios (e.g., intraoperative hemorrhage) tissue ischemia can rapidly cause cellular edema and cytoxicity, especially for patients with predisposing conditions such as diabetes, hypertension, previous intraocular surgery or trauma, ocular infection, retinal detachment, retinal or iris neovascularization and proliferative vitreoretinopathy. As a non-limiting example, hemorrhage encountered during retinal detachment surgery can result in the rapid onset of retinal edema and retinal contraction, making repair of retinal detachments very difficult as the retina shrinks and folds on itself. Cellular death also quickly ensues in such a hemorrhagic event due to oncosis or cytoxic cellular edema, which is driven by hypoxia. The Na/K pump is ATP dependent and therefore does not function properly during hypoxic and oxidative stress. The sodium and ionic influx can cause osmotic influx of water, leading to oncotic cell death. Therefore, intraoperative bleeding can lead to retinal edema and ischemia which ultimately results in poor surgical outcome. The inventors have surprisingly discovered that warming the irrigation fluid to near body temperature so as to allow the coagulation cascade to function at an optimal temperature can prevent or reduce intraoperative bleeding.

The inventors have recognized that during intraoperative bleeding (e.g., vitreous hemorrhage from the retina, choroid or the iris) it is important to deliver the irrigation fluid at near the body temperature of 37° C. to allow for quicker coagulation time and bleeding. While the infusion pressure of the irrigation fluid may be increased to above physiologic pressure to stop intraoperative hemorrhage, this method will cause even greater ischemia and hypoperfusion to the ocular tissue affected by the hemorrhage.

This disclosure provides for a method of using the intraocular irrigation fluid temperature modulator system described herein to present intraocular irrigation fluid at a selected temperature range to a part of the eye of a subject during an intraocular surgery. In some embodiments, the intraocular surgery can include or exclude: cataract surgery (which can include or exclude phacoemulsion), vitrectomy surgery, glaucoma surgery, corneal surgery and other intraocular surgical procedures in the presence of intraoperative hemorrhage or risk of intraoperative hemorrhage. In some embodiments, the part of the eye can include or exclude the anterior chamber, the posterior chamber, or both.

During intraoperative bleeding such as vitreous hemorrhage from the retina, choroid or the iris, it is important to deliver the irrigation fluid at near the body temperature of 37° C. to allow for quicker coagulation time and bleeding. The conventional method for increasing the irrigation fluid temperature is to increase the infusion pressure of the irrigation fluid to above physiologic pressure to stop intraoperative hemorrhage. This method, however, will cause even greater ischemia and hypoperfusion to the ocular tissue affected by the hemorrhage.

Control of intraocular pressure is crucial for ophthalmological procedures such as traditional intracapsular cataract extraction or drainage operations for glaucoma. Intraocular pressure is normally 16+/−5 mmHg, with a value in excess of 25 mmHg considered dangerous to the patient. Intraocular pressure must be maintained within this normal range to ensure constant corneal curvature and a proper refracting index of the eye, including during ophthalmological procedures. Aqueous humour is a clear fluid with a pH 7.1-7.2, a viscosity of 1.025-1.040 relative to water and low protein, urea and glucose content. Aqueous humour occupies the anterior and posterior chambers of the eye. Common vitrectomy and cataract surgical systems require glass bottles because the bottles can be pressurized during surgery.

Systems

In some embodiments, this disclosure provides for an intraocular irrigation fluid temperature modulator system for delivering ocular fluids to part of the eye of a patient during an ophthalmological procedure, the system comprising: a bottle enclosing an interior volume and comprising an exterior surface having contours, and one or a plurality of heating elements configured to be in thermal contact with the contours of the exterior surface of the bottle. The contours of the exterior surface of the bottle can be a round cylindrical surface, or embossed with practical features (which can include or exclude rough portions or indented portions for easier gripping). The intraocular irrigation fluid temperature modulator system can comprise a means to conform the heating element to the exterior contour of the bottle. The means to conform the heating element to the exterior contour of the bottle can be a reversible glue, a buckle, or a Velcro wrap.

In some embodiments, the intraocular irrigation fluid temperature modulator system can further comprise an intraocular irrigation fluid located within the interior volume of the bottle.

In some embodiments, the intraocular irrigation fluid temperature modulator system can further comprise a gas located within the interior volume of the bottle. The gas can include or exclude: air, sulfur hexafluoride, nitrous oxide, oxygen, nitrogen, or combinations thereof.

In some embodiments, the one or plurality of heating elements of the intraocular irrigation fluid temperature modulator system is a heating tape, or a heating coil. The heating tape or heating coil can be embedded within a polymer, silicone, or fabric. The polymer, silicone, or fabric can further comprise a means to removably attach the heating tape or heating coil to the bottle. In some embodiments, the heating element can be selected from an Omega Polyimide Insulated Flexible heater (Omega Engineering), Omega Silicon flexible heating systems, HSTAT Series Silicone Heating Tape (Briskheat), and the like. In some embodiments, the heating element can be those described in U.S. Pat. Nos. 7,019,262; 4,031,352; 4,962,761; 5,871,526, each of which is herein incorporated by reference in their entirety.

In some embodiments, the bottle of the intraocular irrigation fluid temperature modulator system can comprise glass, metal, or a combination thereof. The glass can be surgical grade borosilicate, silica, or pyrex. The purpose of the bottle is to be non-compressible, in that the bottle material will not bend or deform during an ophthalmological procedure when irrigation fluid is removed from said bottle. In some embodiments, the bottle will further comprise one or a plurality of openings. One of the openings in the bottle can be a vent port, so as to maintain an equilibrium pressure within the gas headspace of the bottle containing an irrigation fluid.

In some embodiments, the bottle can comprise an enclosed glass cylinder with one or a plurality of openings wherein the glass cylinder is surrounded by metal. The metal can serve as a heat-transfer medium to more quickly control the temperature of the entire bottle.

In some embodiments, the intraocular irrigation fluid temperature modulator system can further comprise control electronics. The control electronics can comprise a central processing unit, memory chips, instructions encoded in said memory chips, and interfaces to the one or plurality of heating elements. In some embodiments, the control electronics can further comprise an interface to a thermocouple positioned to measure the temperature of the external surface of the bottle. In some embodiments, the control electronics can further comprise a power source. The power source can be a rechargeable battery pack. The rechargeable battery pack can comprise one or a plurality of rechargeable batteries, of which each can independently be selected from a nickel-cadmium (NiCd), nickel-metal hydride (NiMH), lithium-ion (Li-ion), lithium-ion polymer (LiPo), or rechargeable alkaline batteries. In some embodiments, the battery-powered heating element can be those described in U.S. Pat. No. 4,273,989, herein incorporated by reference.

In some embodiments, the rechargeable battery pack can comprise a port for recharging the battery power. The port can be a wireless charger or a USB (Universal Serial Bus) connection. The USB connection can be USB-A, USB-B, USB-C, Micro-USB, mini-USB, and the like.

The control electronics can modulate the voltage and/or current applied to the one or plurality of heating elements so as to modulate the measured temperature within a selected range. In one embodiment, the temperature of the irrigation fluid is modulated to a selected temperature range by applying voltage and/or current to a heating element in thermal contact with the irrigation bottle. The pressure and fluidics of an irrigation delivery system to the eye will not be affected because there is no direct contact of the irrigation delivery system to the irrigation fluid. In some embodiments, the heating element is configured to be positioned at a selected distance from the one or plurality of openings on the bottle so as to reduce contamination and infection risk. In some embodiments, the selected temperature range to set the heating element to can be from 3° C. to 8° C., from 4° C. to 10° C., from 8° C. to 12° C., from 7° C. to 15° C., from 14° C. to 18° C., from 17° C. to 20° C., from 18° C. to 23° C., from 19° C. to 24° C., from 20° C. to 25° C., from 24° C. to 28° C., from 25° C. to 30° C., from 26° C. to 30° C., from 29° C. to 34° C., from 33° C. to 36° C., from 34° C. to 39° C., from 37° C. to 40° C., from 39° C. to 43° C., or any temperature range between any of the aforementioned temperature ranges. In some embodiments, the temperature of the irrigation fluid can be about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, or 43° C.

In some embodiments, the control electronics further can further comprise a receiving device which receives a wireless signal from a control emitter device which transmits a wireless signal and identifies the selected temperature range to which the heating element is to modulate. The selected temperature to set the heating element to can be from 3° C. to 8° C., from 4° C. to 10° C., from 8° C. to 12° C., from 7° C. to 15° C., from 14° C. to 18° C., from 17° C. to 20° C., from 18° C. to 23° C., from 19° C. to 24° C., from 20° C. to 25° C., from 24° C. to 28° C., from 25° C. to 30° C., from 26° C. to 30° C., from 29° C. to 34° C., from 33° C. to 36° C., from 34° C. to 39° C., from 37° C. to 40° C., from 39° C. to 43° C., or any temperature range between any of the aforementioned temperature ranges. In some embodiments, the temperature of the irrigation fluid can be about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, or 43° C.

In some embodiments, the control electronics can further comprise a thermometer or temperature display to indicate about the temperature measured by the thermocouple.

In some embodiments, the control electronics can further comprise a means to electronically disconnect and optionally reconnect, the applied voltage and/or current to the heating element. The means to electronically disconnect and optionally reconnect the applied voltage and/or current to the heating element can be selected from a manual trigger button, a switch, or a relay. In some embodiments, the control electronics can further comprise an audio alarm which activates when the measured temperature is outside of the selected temperature range. In some embodiments, the control electronics can further comprise an electronic circuit which measures the temperature and either applies voltage and/or current to the heating element when the measured temperature is less than a selected temperature range, or applies no voltage and/or current to the heating element when the measured temperature is higher than a selected temperature range. The electronics control system can be configured to receive input from a plurality of temperature sensors. The electronics control system can electronically disconnect the power supply source from the heating element when the plurality of temperature sensors indicates a difference in temperature when the difference between any two temperature sensors is greater than a selected temperature difference.

In some embodiments, the intraocular irrigation fluid temperature modulator system can comprise an insulator material which is configured to be around a portion or all of the exterior surface of the bottle. In some embodiments, the intraocular irrigation fluid temperature modulator system can comprise a transparent window to allow direct visualization of the contents of the bottle.

In some embodiments, the intraocular irrigation fluid temperature modulator system is connected to a vitrectometry system to provide modulated temperature irrigation fluid to the eye of a subject during surgery when the surgeon is using said vitrectometry system. In some embodiments the vitrectometry system is selected from: Alcon Constellation Vision System (Alcon), Stellaris PC (Bausch and Lomb), VersaVit 1.0 or 2.0 (Synergetics), or the EVA (Dutch Ophthalmic (DORC)). The Alcon Constellation Vision System involves a vented gas forced infusion of the irrigation fluid with an attempt to compensate for intraocular pressure. The vented gas forced infusion requires the use of a glass bottle because presented gas into the bottle which pushes fluid out of the irrigation bottle. In some embodiments, the irrigation fluid travels from the irrigation bottle through plastic tubing, into the ocular surgery instrument (e.g., phaco needle) and finally into the anterior chamber of the eye. In some viotrectometry systems, the fluid inflow is based on gravity fluidics, and the infusion pressure is directly related to the irrigation bottle height. To create a pressure gradient the bottle is placed at a height above the patient. Operating a valve opens the irrigation fluid such that it becomes in fluidic communication with a part of the eye of a subject through tubing. In some embodiments, the part of the eye of a subject during an ophthalmological surgery is the anterior chamber. Common fluid delivery systems produce about 11 mm Hg pressure (above ambient atmospheric pressure) intraocular for every 15 cm (6 inches) the bottle height is above the patient's eye. In some embodiments, forced infusion pumps that maintain a preset intraocular pressure during surgery are used to deliver irrigation fluid the anterior chamber of the eye during surgery, with the attempt at providing stable anterior chamber intraocular pressure. In some embodiments, the bottle height or intraocular pressure is set such that the intraocular irrigation fluid infusion pressure is adequate to balance the outflow of such fluid. This pressure balance attempts to maintain a stable anterior chamber by keeping the pressure in the anterior chamber approximately constant during surgery. However, if the balance of inflow and outflow is altered, for example by increasing the fluid delivery pressure to stop intraoperative hemorrhage, the anterior chamber can become under or over-pressurized. Under-pressurization of the anterior chamber of the eye can lead to shallowing and/or collapse on the anterior chamber, resulting in forward movement of the iris, lens and posterior capsule. In addition, under-pressurization may lead to inadvertent rupture of the posterior capsule due to its movement towards the surgery instrument. Over-pressurization of the anterior chamber of the eye (bottle height or intraocular pressure set too high) can cause misdirection of aqueous fluid or deepening of the anterior chamber with zonular stress.

Conventional inline surgical fluid warming systems with heat exchangers applied to the delivery tubing disrupt the air pressure input line to the surgical fluid warming reservoir, resulting in uncontrolled pressure regulation of the intraocular irrigation solution presented to the eye. Modification of conventional inline surgical fluid warming systems by separating the air line from the fluid outflow line (currently they are bundled together and have one single spike into the bottle) and use of an in-line warming cassette would require priming of the entire fluid line with minimal control over the backpressure from the extra tubing length and/or the reduction in tubing diameter when the delivery line is connected to the chambers of the eye. In addition, to heat up the irrigation fluid during high flow rate (the case during surgical bleeding), would require multiple loops through a warming cassette, and a means to increase the air pressure to compensate for the added backpressure. The introduction of additional tubing length presents the risk that a “kink”, or partial blockage of the tubing may form, causing disruption and even stoppage of fluid flow. If there is a kink in the soft tubing that is embedded in a warming cassette, it would be difficult to unkink during surgery. In fluid dynamics, liquids flowing through tubes will experience back-pressure from the friction of the fluid with the surface of the tubing. Twists, bends, and partial blockages in the tubing will increase back-pressure. Warming cassettes which are conventionally used to increase fluid temperature rely on multiple loops of tubing passing through a thermal transfer area will increase back-pressure of the tubing. Back-pressure in tubing makes it difficult to control the delivered ocular fluid at a consistent temperature and fluid. The present systems and methods, however, do not involve multiple bent tubing for thermal transfer and therefore avoid back-pressure created by twists and turns in fluid warming cassettes. The present systems and methods enable the delivery of ocular fluids at a consistent pressure and temperature. Thus, it is highly problematic to modulate the temperature to a selected temperature range of intraocular irrigation fluids using conventional inline irrigation surgical delivery lines.

Furthermore, conventional inline irrigation surgical delivery systems involving an inline tubing cassette would result in a high cost and high rate of disposal of unused tubing cassettes, for example the cassettes described in U.S. Pat. Nos. 9,498,586; 5,514,094, 5,381,510; 5,729,653; 5,733,263; 7,031,602; 5,420,962; 4,735,609; 6,824,528; U.S. Pat. App. No. 20050244512, and U.S. Pat. App. No. 20080161773. Typically, only about 10-20% of ophthalmological surgeries require warmed infusion fluid because most ophthalmological surgeries do not present increased bleeding risks. Thus, conventional intraocular irrigation fluid warming systems which are not modular, and require fixed infrastructure, would add the burden of the heating infrastructure to all ophthalmological surgeries, including those which would not require intraocular irrigation fluid warming. In addition, conventional intraocular irrigation fluid delivery systems would require complex electronics to determine when the intraocular irrigation fluid warming system were to be engaged.

The intraocular irrigation temperature modulator system of the present invention, in contrast, includes the advantages that it is relatively inexpensive, can be selectively utilized, and does not interfere with the irrigation flow rate.

### EXAMPLES

### Example 1

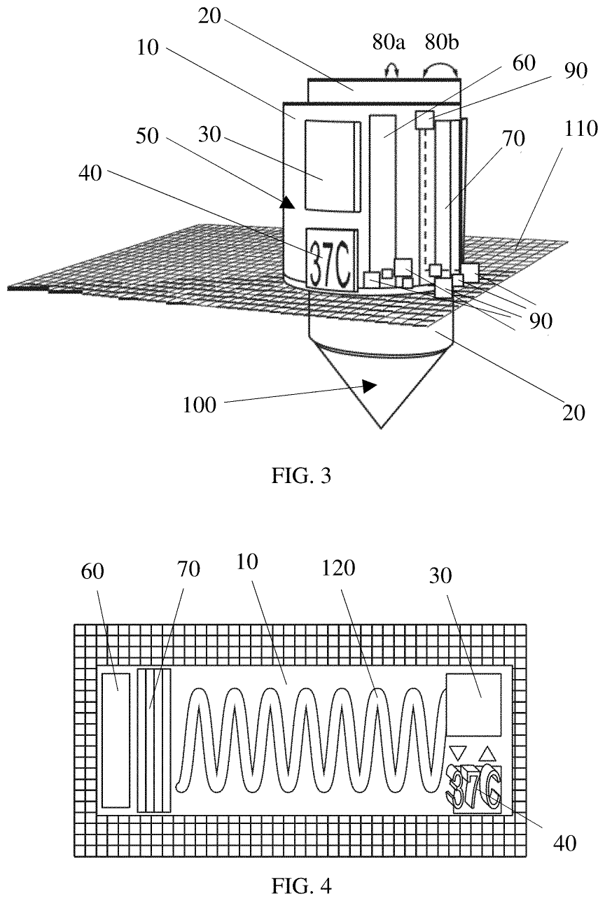

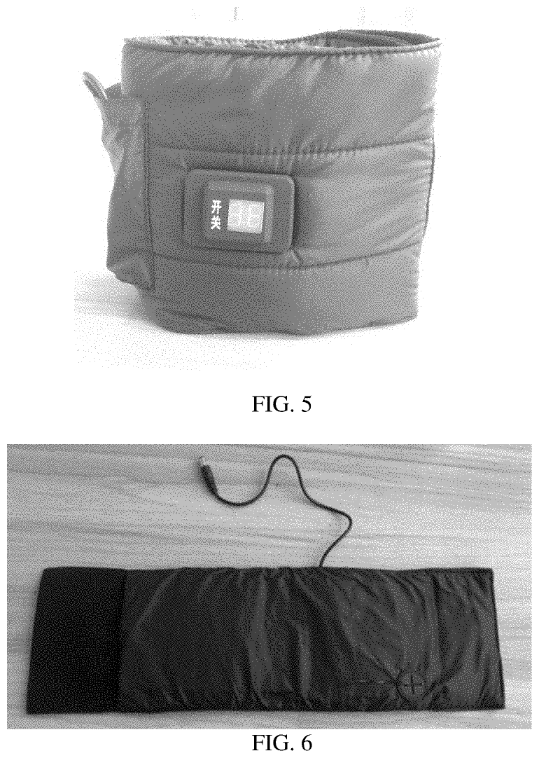

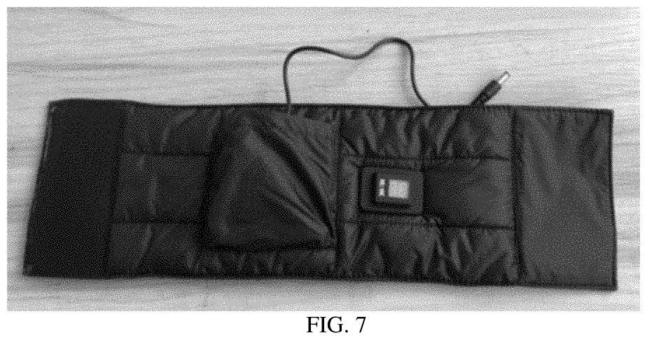

The construct of the heated band is analogous to an electrically heated blanket or garment. As a non-limiting example for a band that wraps around a 500 ml irrigation bottle, the approximate length and width of the bottle would be 45 cm×17 cm. The band would be made of water-resistant fabric. Inside the band would be layers of insulation made from cotton or other insulation material in the ideal embodiment. Referring to FIG. 3 and FIG. 4, the heating element would consist of heating coils or wires embedded within the band in a winding pattern to maximize heat delivery. A preferred embodiment of the heating element would be silicone rubber heating element where current passes to generate heat. It would preferably run at 5-12V DC. The preferred battery pack would be a Lithium ion battery pack generating 7.4V DC and max 2.5 A and the battery pack would be enclosed within the band in a pocket so that the battery is protected from fluid splashes. The preferred securing device for the band would be a Velcro strip, buckle or snap-on button to secure the band around the bottle tightly. The electronic control system would be embedded within the band. The electronic control system would employ feedback from the plurality of temperature sensors to achieve a target temperature setting, ideally around body temperature. There would be a water-proof on-off button and temperature adjustment button and a temperature display visible to the user. A preferred embodiment would also include a clear plastic or acrylic window to enable direct visualization of the content of the bottle, including bubbles and remaining fluid level. An audible and visual alarm system would be included in the preferred embodiment in case of over- or under-heating, low battery or system malfunction.

It will be understood that the foregoing description is of exemplary embodiments of this invention, and that the invention is not limited to the specific forms shown. Modifications may be made in the design and arrangement of the elements without departing from the scope of the invention.

The inventions described and claimed herein have many attributes and embodiments including, but not limited to, those set forth or described or referenced in this Detailed Disclosure. It is not intended to be all-inclusive and the inventions described and claimed herein are not limited to or by the features or embodiments identified in this Detailed Disclosure, which is included for purposes of illustration only and not restriction. A person having ordinary skill in the art will readily recognize that many of the components and parameters may be varied or modified to a certain extent or substituted for known equivalents without departing from the scope of the invention. It should be appreciated that such modifications and equivalents are herein incorporated as if individually set forth. The invention also includes all of the steps, features, compositions and compounds referred to or indicated in this specification, individually or collectively, and any and all combinations of any two or more of said steps or features.

All patents, publications, scientific articles, web sites, and other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety. Applicants reserve the right to physically incorporate into this specification any and all materials and information from any such patents, publications, scientific articles, web sites, electronically available information, and other referenced materials or documents. Reference to any applications, patents and publications in this specification is not, and should not be taken as, an acknowledgment or any form of suggestion that they constitute valid prior art or form part of the common general knowledge in any country in the world.

The specific methods and compositions described herein are representative of preferred embodiments and are exemplary and not intended as limitations on the scope of the invention. Other objects, embodiments, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. Thus, for example, in each instance herein, in embodiments or examples of this, any of the terms “comprising”, “consisting essentially of”, and “consisting of” may be replaced with either of the other two terms in the specification. Also, the terms “comprising”, “including”, containing”, etc. are to be read expansively and without limitation. The methods and processes illustratively described herein suitably may be practiced in differing orders of steps, and that they are not necessarily restricted to the orders of steps indicated herein or in the claims. It is also that as used herein and in the appended claims, the singular forms “a,” “an,” and “the” include plural reference unless the context clearly dictates otherwise. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants. Furthermore, titles, headings, or the like are provided to enhance the reader's comprehension of this document, and should not be read as limiting the scope of this. Any examples of embodiments, embodiments or components of the invention referred to herein are to be considered non-limiting.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although this has been specifically disclosed by preferred embodiments and optional features, modification and variation of the concepts herein disclosed may be resorted to by those skilled in the art, and that such modifications and variations are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or embodiments of the invention are described in terms of Markush groups, those skilled in the art will recognize that the invention is also thereby described in terms of any individual member or subgroup of members of the Markush group.

What is claimed:

1. An intraocular irrigation fluid temperature modulator system for delivering ocular fluids to part of the eye of a patient during an ophthalmological procedure, the system comprising:
a. a bottle enclosing an interior volume and comprising an exterior surface having contours, and
b. one or a plurality of heating elements configured to be in thermal contact with the contours of the exterior surface of the bottle.

2. The intraocular irrigation fluid temperature modulator system of claim 1, further comprising an intraocular irrigation fluid located within the interior volume of the bottle.

3. The intraocular irrigation fluid temperature modulator system of claim 1, wherein the one or plurality of heating elements is a heating tape.

4. The intraocular irrigation fluid temperature modulator system of claim 1, wherein the heating element is a heating coil.

5. The intraocular irrigation fluid temperature modulator system of claim 1, wherein the heating element is embedded within a polymer, silicone, or fabric.

6. The intraocular irrigation fluid temperature modulator system of claim 1, wherein the bottle comprises glass, metal, or a combination thereof.

7. The intraocular irrigation fluid temperature modulator system of claim 6, wherein the bottle is non-compressible.

8. The intraocular irrigation fluid temperature modulator system of claim 6, wherein the bottle comprises an enclosed glass cylinder with one or a plurality of openings.

9. The intraocular irrigation fluid temperature modulator system of claim 1, further comprising a gas located within the interior volume of the bottle.

10. The intraocular irrigation fluid temperature modulator system of claim 9, wherein the gas is selected from air, sulfur hexafluoride, nitrous oxide, oxygen, nitrogen, or combinations thereof.

11. The intraocular irrigation fluid temperature modulator system of claim 1, further comprising a means to conform the heating element to the exterior contour of the bottle.

12. The intraocular irrigation fluid temperature modulator system of claim 1, wherein the means to conform the heating element to the exterior contour of the bottle is selected from a reversible glue, a buckle, or a Velcro wrap.

13. The intraocular irrigation fluid temperature modulator system of claim 1, further comprising control electronics which comprises: a thermocouple positioned to measure the temperature of the external surface of the bottle; a thermometer or temperature display to indicate about the temperature measured by the thermocouple; a means to electronically disconnect and optionally reconnect, the applied voltage and/or current to the heating element;
wherein the control electronics is configured to modulate the voltage and/or current applied to the heating element so as to modulate the measured temperature obtained from the thermocouple, within a selected range by applying voltage and/or current to the heating element when the measured temperature is less than a selected temperature range, or applying no voltage and/or current to the heating element when the measured temperature is higher than a selected temperature range.

14. The intraocular irrigation fluid temperature modulator system of claim 13, wherein the control electronics further comprises a receiving device which receives a wireless signal from a control emitter device which identifies the selected temperature range to which the heating element is to modulate.

15. The intraocular irrigation fluid temperature modulator system of claim 13, wherein the means to electronically disconnect and optionally reconnect the applied voltage and/or current to the heating element comprises a manual trigger button.

16. The intraocular irrigation fluid temperature modulator system of claim 13, wherein the control electronics further comprises an audio alarm which activates when the measured temperature is outside of the selected temperature range.

17. The intraocular irrigation fluid temperature modulator system of claim 13, wherein the electronics control system is configured to receive input from one or a plurality of temperature sensors.

18. The intraocular irrigation fluid temperature modulator system of claim 14, wherein the electronics control system electronically disconnects the power supply source from the heating element when the plurality of temperature sensors indicates a difference in temperature when the difference between any two temperature sensors is greater than a selected temperature difference.

19. The intraocular irrigation fluid temperature modulator system of claim 1, further comprising a transparent window to allow direct visualization of the contents of the bottle.

20. The intraocular irrigation fluid temperature modulator system of claim 1, wherein the part of the eye is selected from the anterior or posterior chamber of the eye.

21. A method for presenting intraocular irrigation fluid at a selected temperature range to a part of the eye of a subject during an ophthalmological procedure without increasing the inflow pressure, the method comprising: administering to the eye of a patient having an ophthalmological procedure an intraocular irrigation fluid at a selected temperature using the intraocular irrigation fluid temperature modulator system of claim 1.

22. The method of claim 21, wherein the ophthalmological procedure is selected from cataract surgery, vitrectomy surgery, glaucoma surgery, corneal surgery and other intraocular surgical procedures in the presence of intraoperative hemorrhage or risk of intraoperative hemorrhage.

23. The method of claim 22, wherein the part of the eye is selected from the anterior or posterior chamber of the eye.
